(19) United States
(12) Patent Application Publication
(10) Pub. No.: US 2009/0131974 A1
(43) Pub. Date: May 21, 2009

(54) INSTRUMENT, PARTICULARLY FOR USE IN LAPAROSCOPIC SURGERY

(75) Inventors: Terje S. Pedersen, Sandnes (NO); Reza Hezari, Trondheim (NO)

Correspondence Address: KNOBBE MARTENS OLSON & BEAR LLP, 2040 MAIN STREET, FOURTEENTH FLOOR, IRVINE, CA, 92614, US

(73) Assignee: SURGITECH NORWAY AS, Trondheim (NO)

(21) Appl. No.: 11/813,082

(22) PCT Filed: Dec. 23, 2005
(86) PCT No.: PCT/NO2005/000478

§ 371 Date: Sep. 29, 2008

(30) Foreign Application Priority Data
Dec. 29, 2004 (NO) 20045706

(51) Int. Cl. A61B 17/00

(52) U.S. Cl. 606/205

(57) ABSTRACT

A laparoscopic instrument (1) including a grip (2) provided with an actuator (3) which can effect, through a linkage (5), the manipulation of an effector (9) placed at a first end portion of a tubular element (7), the tubular element (7) being attached to the grip (2) of the instrument (1), in which there is arranged a ratchet mechanism, in which a pawl (24) is arranged to cooperate with a substantially complementary ratchet bar (22), teeth of the pawl (24) being arranged to be activated or deactivated for engaging teeth of the ratchet bar (22), by means of a connection switch (26) and the connection switch (26) being connected to the pawl (24) of the ratchet mechanism (20) via a flexible element (28).

This invention relates to an instrument, in particular an instrument for use in laparoscopic surgery, also called “keyhole surgery”.

The object of the invention is to provide a simple manually operated instrument in which the tool or so-called effector of the instrument is arranged to be selectively locked in a desired position and to be released from the locked position without an ideal finger grip having to be shifted for the instrument to be operated. An ideal finger grip may be for example, but not exclusively, a three finger grip in which the little finger, the ring finger and the middle finger enclose a portion of the grip of the instrument, and in which the index finger, first finger, is used to operate the main function of the effector of the instrument, such as a pair of scissors or tongs. This gives the operator; a surgeon for example, greatly improved control of the instrument.

Even though laparoscopy was carried out for the first time on a human being in 1910, it was not until 1987 that the use of laparoscopic techniques took off. Since then there has been a violent process of change in areas of use and surgical procedures. However, the development of laparoscopic instruments has been minimal with respect to ergonomic improvements. Scientific measurements go to show that a surgeon expends up to ten times the amount of energy to carry out the same procedure laparoscopically compared with open surgery.

A great number of designs of laparoscopic instruments are known from the U.S. Pat. Nos. 5,480,409, 5,893,878, 5,383,888, 5,792,165, 5,976,121, 5,488,441, 5,735,873 and 5,868,784 and from WO 9724072. Even though the known instruments vary greatly in design and function, the known instruments have in common that they are constituted by a grip including one or more movable parts, a “trigger” among other things, which can be manipulated by the user, for example the surgeon, to control a tool or an effector which is connected to a cantilever portion of a tubular element or tool rod which is connected at its other portion to the grip.

U.S. Pat. No. 5,792,165 discloses an instrument exhibiting great flexibility with respect to the manoeuvring of an effector, which has three liberties: rotation, pivoting and clamping. In addition different effectors may be connected to and removed from the tubular body of the instrument. The instrument disclosed in U.S. Pat. No. 5,792,165 may also be provided with an integrated motor and microprocessor partially controlling the actions of the effector.

U.S. Pat. No. 5,383,888 discloses an instrument exhibiting essentially the same functions as the instrument of U.S. Pat. No. 5,792,165.

U.S. Pat. No. 5,976,121 discloses a grip for manipulating an instrument in connection with endoscopy.

U.S. Pat. No. 5,735,873 discloses a surgical instrument provided with a ratchet mechanism in the grip, and an actuator and effector at the end of the surgical instrument.

U.S. Pat. No. 5,868,784 discloses a surgical instrument provided with a ratchet mechanism arranged to lock an actuator.

WO 9724072 deals with a laparoscopic instrument having an adjustable grip to allow the instrument to be adapted for different hand sizes. The instrument includes locking means for locking the trigger in a desired position.

There are several drawbacks associated with the prior art mentioned above.

One of the drawbacks relates to the very design of the grips of the instruments which are ergonomically unfavourable in the great majority of the above-mentioned prior art techniques, because the instruments do not provide for a volar-flexed working position and/or it is necessary to move fingers to operate the instrument, and other fingers than the index finger will have to be used to operate the main functions of the instrument. This entails that small uncontrolled movements may easily occur in the surgeon's hand portion. These movements lead to relatively large and undesired movements at the operative end portion of the instrument, at which the effector is placed. A result of this unfavourable design is that in an attempt to counteract the above-mentioned undesired movements, among other things, a surgeon expends up to ten times the amount energy to carry out the same procedure laparoscopically compared with so-called open surgery.

Another substantial drawback related to some of the above-mentioned instruments is that they are technically very complex, which entails that the instruments will be expensive to manufacture. Thereby, to a very great degree, the instruments are intended to be reused several times. Even though, theoretically, instruments can be disinfected 1001, the study “The Clinical suitability of laparoscopic instrumentation. A prospective clinical study of function and hygiene.” carried out by Fengler, Pahlke, Bisson and Kraas at the Department of Surgery, Krankenhaus Moabit, Lehrkrankenhaus der Humboldt Universität zu Berlin, among others, shows that after cleaning, a relatively large number of instruments contain residues of blood products, which represent a potential risk of patients being subjected to contagion. This may lead to the patient becoming seriously ill and, at worst, dying.

In connection with laparoscopic surgery there is often the need to be able to lock an effector, such as, but not limited to, a pair of gripping tongs in a particular position in or between the closed and fully open positions. Locking is particularly relevant in connection with the gripping and securing of, for example, tissues or blood vessels. The instruments disclosed in U.S. Pat. Nos. 5,792,165, 5,480,409, 5,735,873, 5,868,784 and WO 9724072 are provided with different forms of ratchet mechanisms that engage each other to lock the effector in a particular position. A substantial drawback of the solutions disclosed in said patent documents is that the engagement and disengagement of the pawl from the ratchet wheel must be carried out by one or more fingers or the whole hand having to be moved from its natural position on the trigger used to control the effector. U.S. Pat. No. 5,868,784 discloses a very complicated instrument, which is provided with a ratchet mechanism, which locks the trigger in the desired position. A substantial drawback of said patent and of the patents WO 9724072 and U.S. Pat. No. 5,735,873 is that the trigger is locked against any movement after the ratchet mechanism has been activated.

The present invention has as its object to remedy or at least reduce one or more of the drawbacks related to the prior art represented by the above-mentioned patent documents, and then in particular the drawbacks related to the functionality of the mechanism locking the effector in a desired opening position. At the same time it is an object to provide an instrument which both exhibits a very simple construction and in which a substantial part of the components of the instrument can be produced of for example, but not limited to, plastics materials. This leads to relatively low production costs and could thereby defend the use of the instrument as a disposable item. This, again, will eliminate the problem of contagion being transmitted due to inadequate cleaning of the instrument.

The object is achieved in accordance with the invention through the features specified in the description below and in the following Claims.

In one aspect the present invention is constituted by an instrument including a grip which is provided with an actuator which is arranged to effect, via a linkage, the manipulation of an effector placed at a first end portion of a tubular element, said tubular element extending at its second end portion into the grip of the instrument and being connected to a portion of the linkage, the linkage including a ratchet bar which is arranged to cooperate with a substantially complementary pawl, slanting teeth of the pawl being arranged to be activated or deactivated for engagement with slanting teeth of the ratchet bar by means of a connection switch, the connection switch being connected to the pawl through a flexible element which is arranged to bias the pawl into engagement with the ratchet bar when the connection switch is in a first position, and which is arranged to apply a pull force on the pawl when the connection switch is in a second position, the pawl being arranged to be disengaged from the ratchet bar only when the pull force from the flexible element at least exceeds an opposite resultant of the frictional forces occurring between the slanting teeth of the pawl and ratchet bar and able to maintain said engagement even after the connecting switch has been moved from the first position into the second position.

In a preferred embodiment the slanting complementary teeth of the pawl and ratchet bar allow one-way relative movement of the ratchet bar relative to the pawl. Therefore, as mentioned above, the combination of slanting teeth and the flexible element will allow the pawl to disengage from the ratchet bar only when the connection switch is in a non-activated position and the ratchet bar is moved relative to the pawl, which is achieved, according to the present invention, through a movement of the actuator as the ratchet bar is preferably arranged in connection with the linkage. It will thus be understood that the connection switch for the ratchet mechanism can be controlled independently of the actuator and that the ratchet mechanism is disconnected by means of the actuator. This is an important property to achieve full control of the instrument. A corresponding effect could not be achieved if the flexible element was replaced by a substantially rigid element.

In order further to ensure that the ratchet mechanism will not be disengaged uncontrolledly after the connection switch has been moved into the non-activated position, the linkage is provided, in a preferred embodiment, with a biasing element causing full engagement between the preferably slanting teeth of the pawl and ratchet bar.

In a preferred embodiment said tubular element and the effector placed at the first end portion thereof are arranged to be rotated about the longitudinal axis of the tubular element and relative to the grip, by means of a rotary wheel of a type known per se.

A laparoscopic operation may last for a relatively long time. Therefore, it is very important that the instrument has the best possible adjustment to the operator's hand, both with respect to the positioning of functional devices like the actuator and connection switch, but also with respect to size. Therefore, in a preferred embodiment the instrument according to the present invention is provided with a grip which is provided with at least one adjustable portion enabling adjustment of the grip to the size of the instrument operator's hand. In one embodiment the at least one adjustable portion of the instrument is placed in a back portion of the grip.

In what follows, there is described a non-limiting example of a preferred embodiment which is visualized in the accompanying drawings, in which:

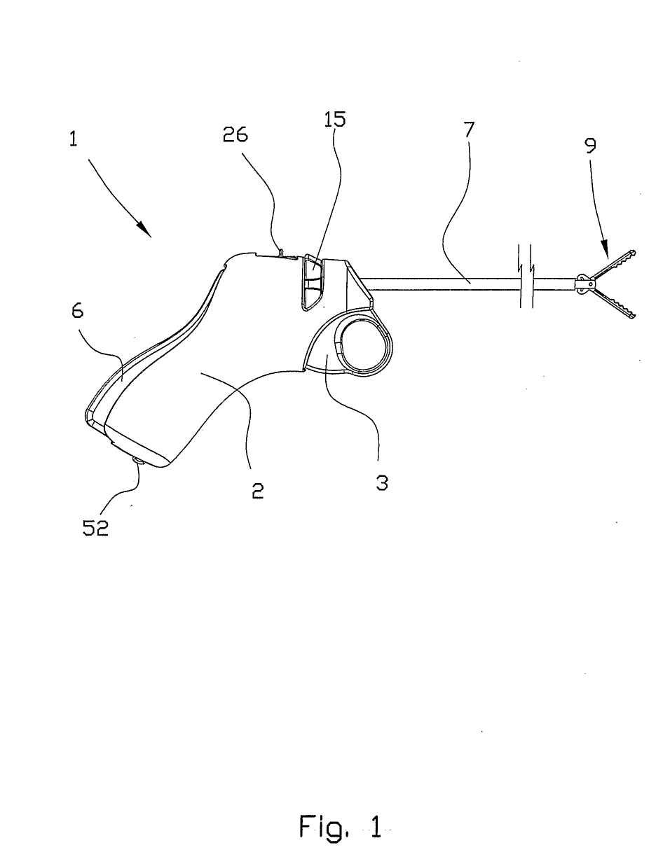

FIG. 1 shows a view of a laparoscopic instrument in accordance with the invention, in which an actuator in the grip of the instrument is in an initial position, in which the actuator is not subjected to an external force, and in which an effector in the form of a gripping claw placed at the first end portion of a tubular element is in an open position. A connection switch at the upper portion of the grip is in a first or non-activated position.

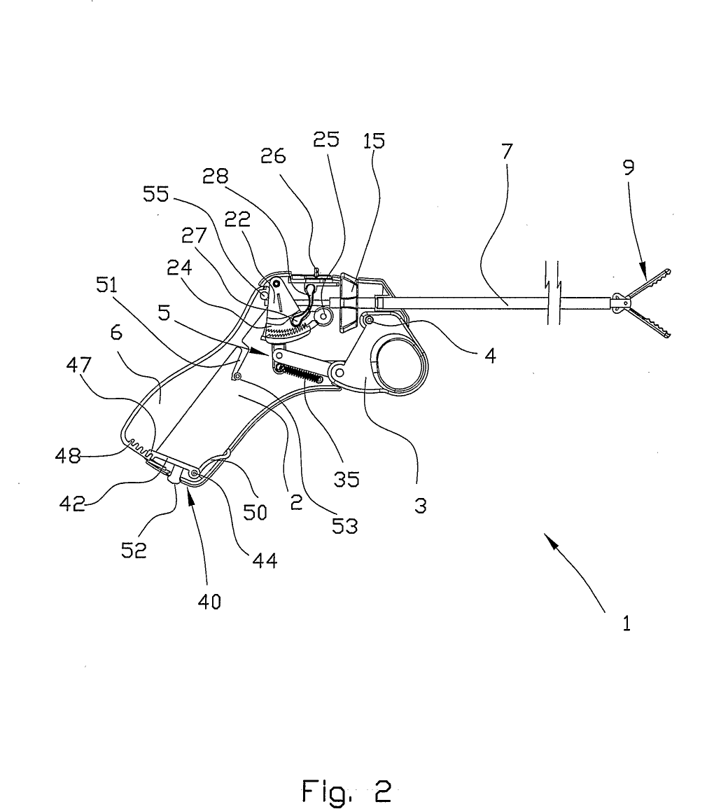

FIG. 2 shows a view of the instrument of FIG. 1, a cover having been removed from the grip.

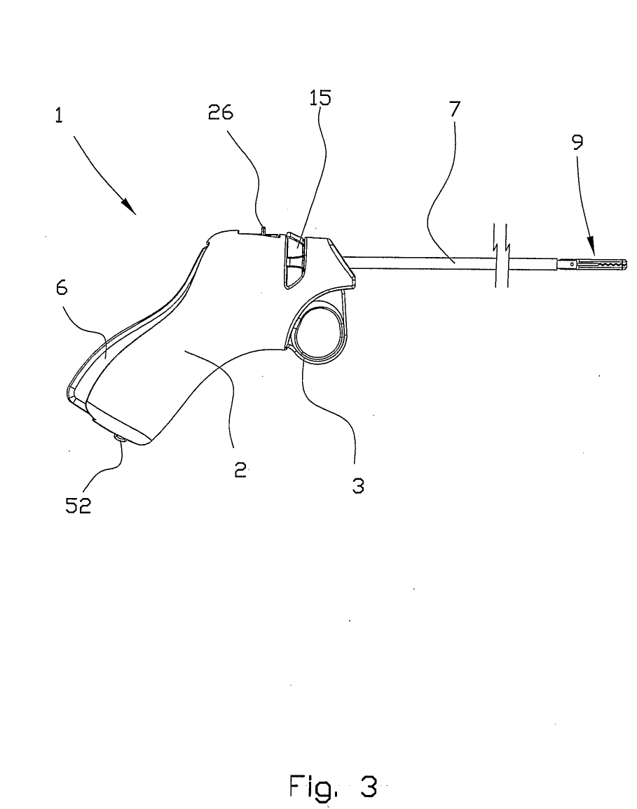

FIG. 3 shows the instrument in FIG. 1 after an external force has been applied to the actuator and moved it to a depressed or activated position, the gripping claw thereby being in a closed position.

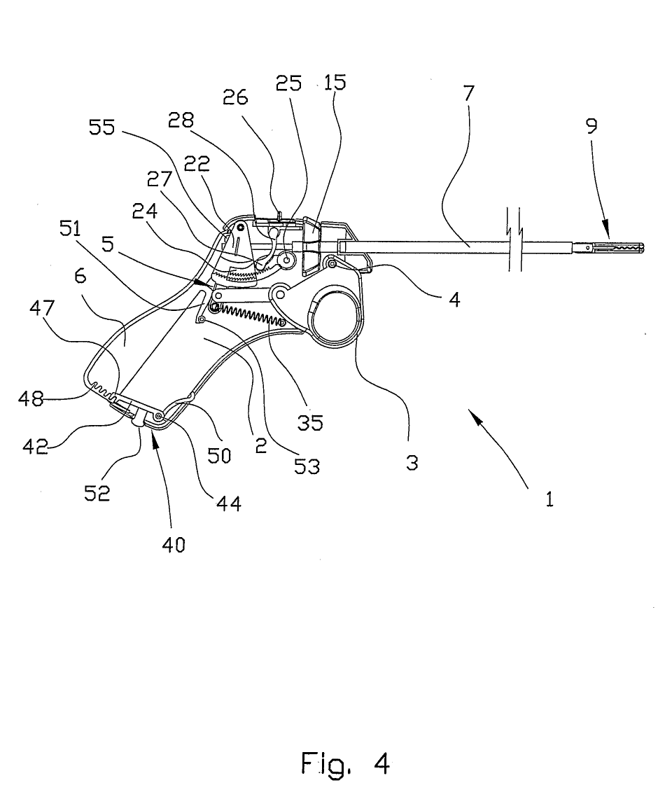

FIG. 4 shows a view of the instrument of FIG. 3, a cover having been removed from the grip.

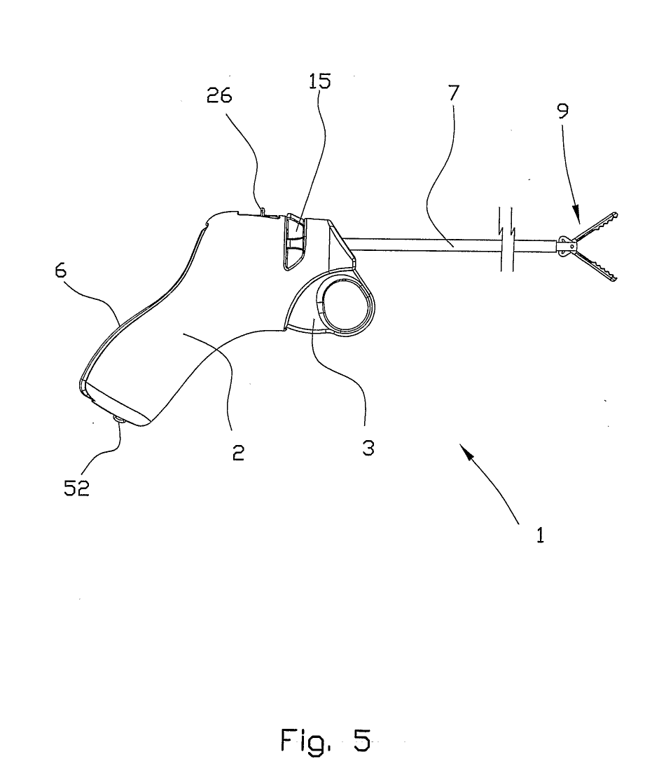

FIG. 5 shows the instrument of FIG. 1 after an adjustable back portion has been placed in its near-innermost position.

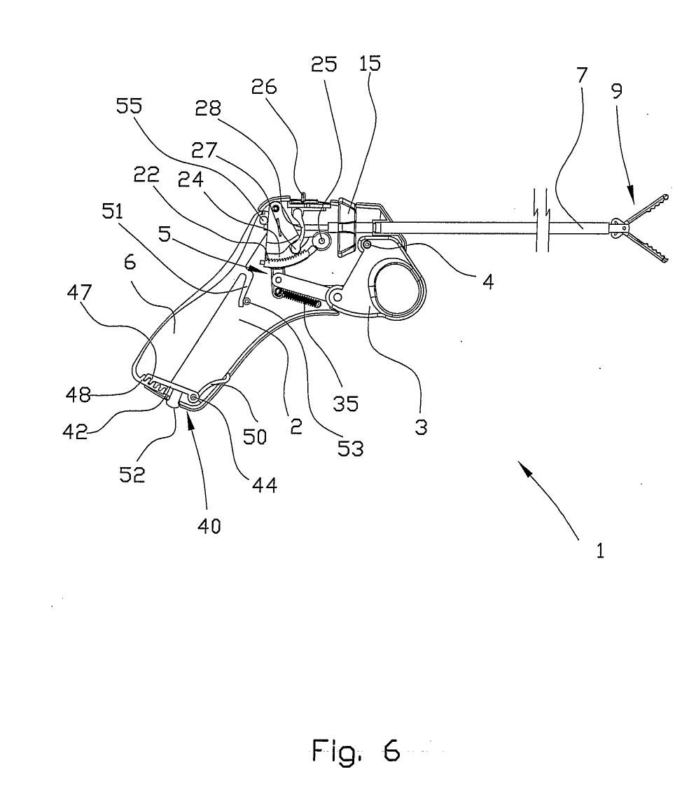

FIG. 6 shows a view of the instrument of FIG. 5 with a cover removed, but with the connection switch moved to an activated position and, because of that, the pawl of the ratchet mechanism being biased into engagement with the ratchet bar of the ratchet mechanism.

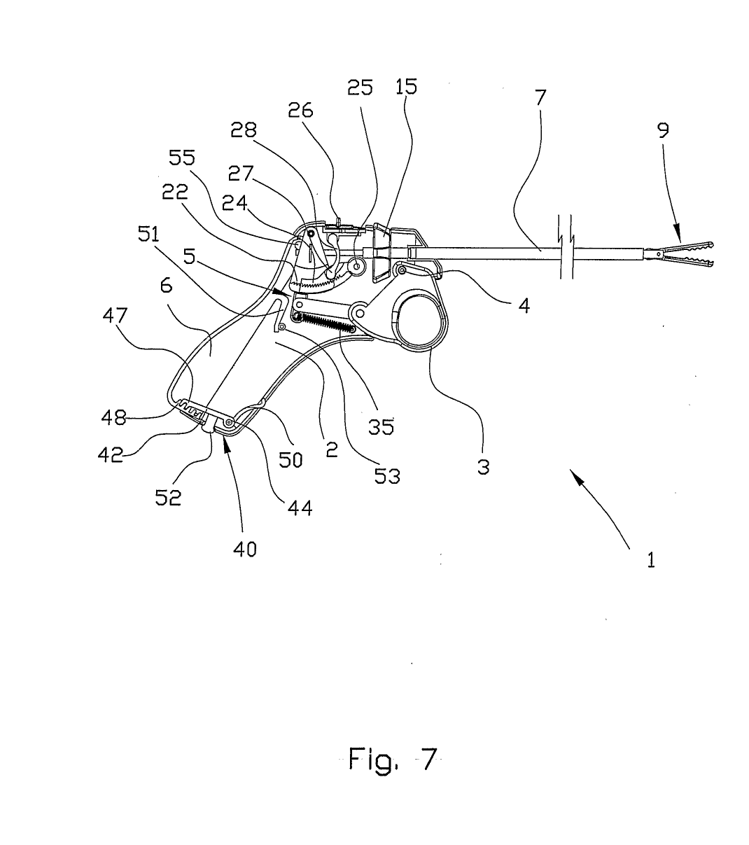

FIG. 7 shows a view of the instrument of FIG. 6, the actuator having partially been moved into the grip and the gripping claw being partially closed.

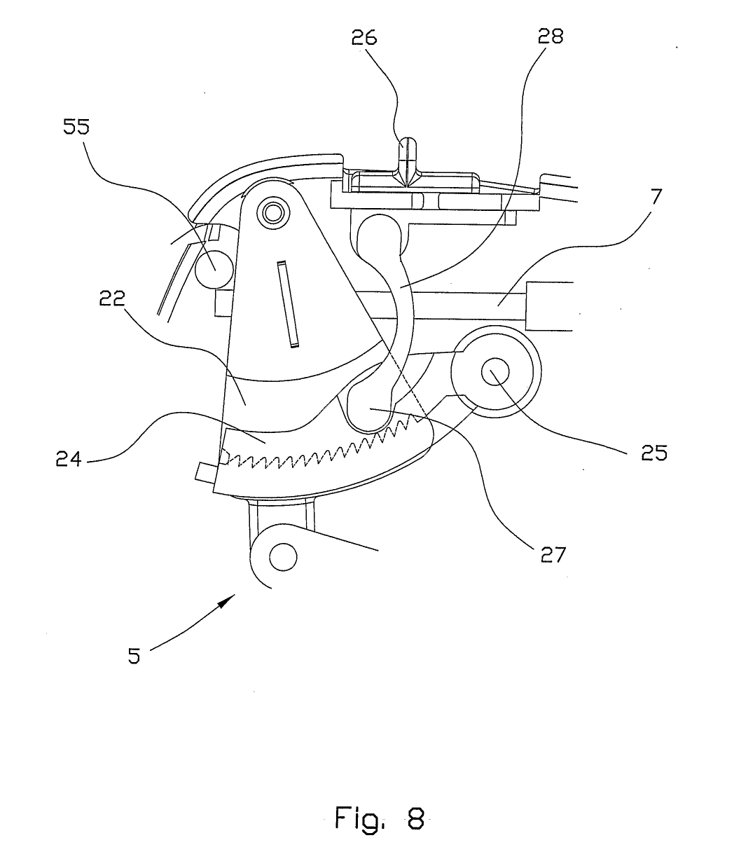

FIG. 8 shows, on a larger scale, a side view of a portion of the instrument of FIG. 5, in which the connection switch is in the activated position and in which the flexible element has driven the pawl into engagement with the ratchet bar. For clarity the teeth of the pawl and ratchet bar have are shown partially visible by means of broken lines.

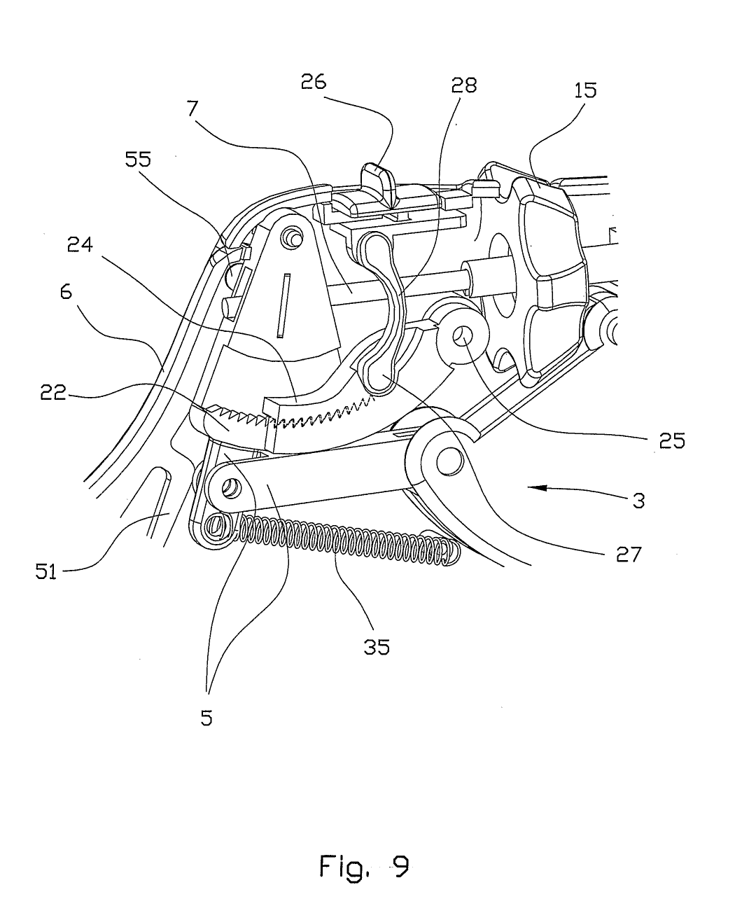

FIG. 9 shows, on a larger scale, a perspective view of a portion of the instrument as the connection switch is in the activated position and the actuator has been moved to a substantially fully depressed position.

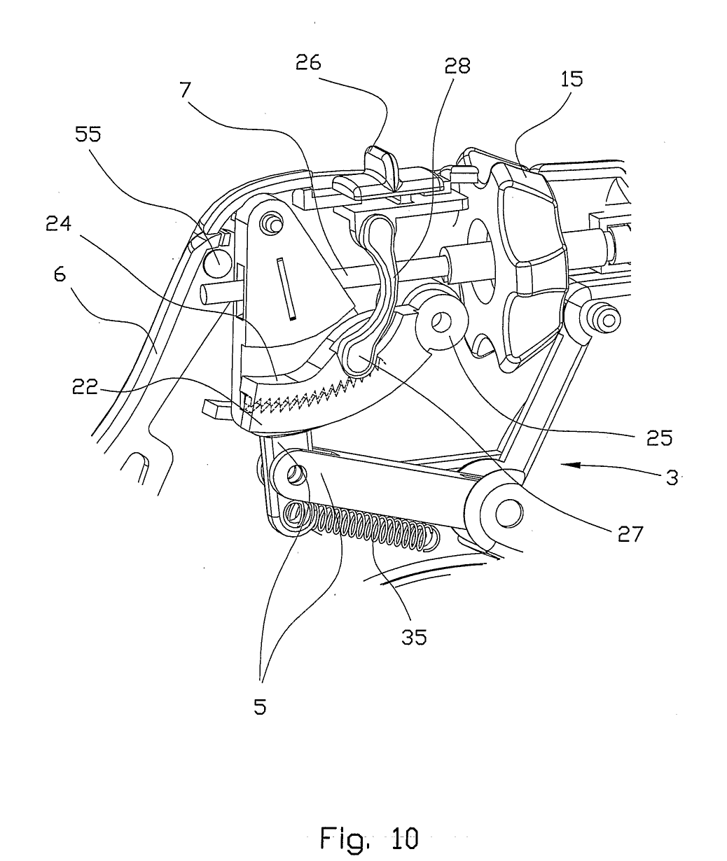

FIG. 10 shows a perspective view of a portion of the instrument, in which the connection switch is in a non-activated position, but in which the teeth of the pawl still engage the teeth of the ratchet bar and the flexible element that connects the connection switch and the pawl is subjected to tensile forces.

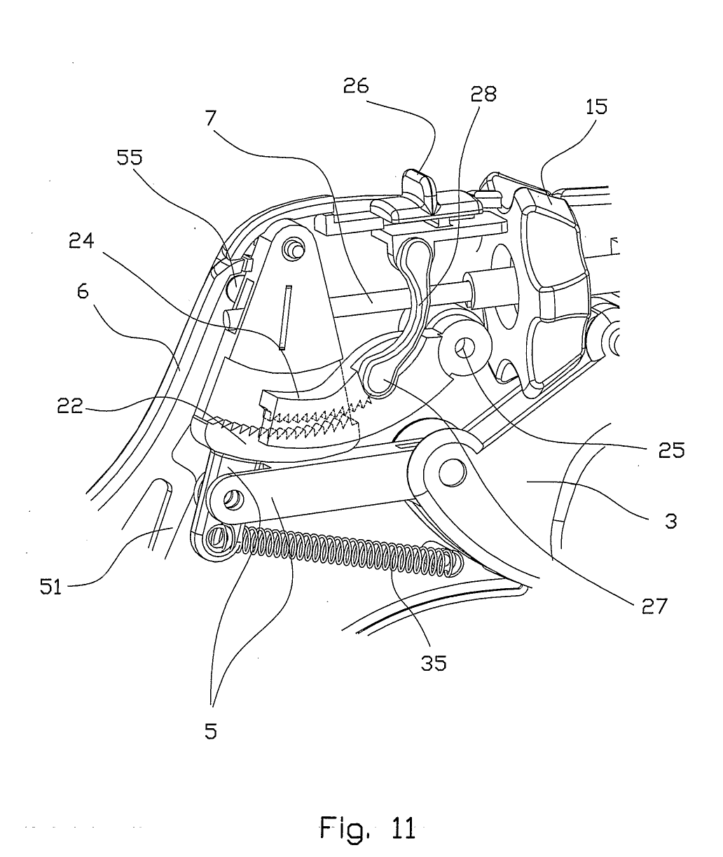

FIG. 11 shows a perspective view of a portion of the instrument of FIG. 4, in which the connection switch is in a non-activated position, and in which the actuator has been moved to a substantially fully depressed position and in which the pawl is disengaged from the ratchet bar.

In the drawings the reference numeral 1 indicates a laparoscopic instrument which is constituted by a grip 2 which is provided with a trigger or actuator 3 which is rotated, when subjected to an external force (not shown), about an axle 4, and in which the actuator 3 is connected via a linkage 5 to a tubular element 7 of a kind known in itself. The tubular element 7 is placed in such a way that it projects from the grip 2. At its first end portion the tubular element 7 is provided with an effector 9 in the form of needle-nose pliers, which can be used in laparoscopic surgery, for example.

The tubular element 7 is rotatable about its longitudinal axis. The rotation is controlled by means of a rotary wheel 15 placed in the upper portion of the grip 2. The effector 9 is rotated together with the tubular element 7.

The jaw of the effector 9 is arranged to be locked in a desired position by activation of a ratchet mechanism constituted by a curved cogged ratchet bar 22 and a substantially complementary, cogged pawl 24. For clarity, the ratchet mechanism of FIGS. 2, 4, 6, 7 and 8-11 is shown “transparently”, so that the entire sets of teeth of the ratchet bar 22 and pawl 24 are shown.

In the exemplary embodiment, the ratchet bar 22 is shown as an integral part of the linkage 5.

At a first end portion, the pawl 24 is rotatably attached to the grip 2 by means of an axle 25, and is rotatably connected by way of a flexible element 28 to a movable connection switch 26 placed in the upper portion of the grip 2. The flexible element 28 is attached to the pawl 24 at a point of attachment 27 placed between said axle 25 and a second, free end portion of the pawl.

The tubular element 7 is fixed to the rotary wheel 15, and the effector 9 is rotationally fixed to the tubular element 7. By rotating the wheel 15, said tubular element 7 and the effector 9 will be rotated relative to the grip 2 in a manner known per se.

In the FIGS. 1 and 2 the actuator 3 is unloaded by external forces and is in its fully open position. The actuator 3 is urged into this position by a biasing element in the form of a spring 35 connected between the linkage 5 and a portion of the grip 2 in such a way that the biasing force is transmitted via the linkage 5 as a compressive force on the actuator 3 and causes this to be rotated about the axle 4 into its initial position which is the furthest possible away from the grip, a position which will be referred to below as the non-activated position. When the actuator 3 is non-activated, the effector 9, which is placed in the first end portion of the tubular element 7, is in its fully open position. A person skilled in the art will appreciate that in an alternative embodiment the effector 9 may be in its fully closed position when the actuator 3 is in its non-activated position.

The instrument 1 is provided with an adjustable back portion 6, which is shown in FIGS. 1-4 in its most projecting position. The back portion 6 is locked in the position by means of a locking member 40, which is formed by a locking element 42, which is rotatably connected at its first end portion to a portion of the grip 2 in an attachment portion 44. At its second end portion the locking element 42 is provided with a claw member 47 which is arranged to grip across one of several (five are shown) dogs 48 complementary to the claw member 47, which are placed in and project from an internal bottom portion of the back portion 6. The locking element 42 is biased by means of a spring member 50 in such a way that the claw 47 is moved to bear on the dog 48. In a desired adjustment of the back portion 6 an adjustment button 52 is subjected to a force counteracting the force of the spring member 50, thereby disengaging the claw 47 from the dog 48. When the claw 47 is disengaged from the dog 48, the back portion 6 will be biased into its outermost position by a biasing element 51 projecting from an internal portion of the back portion and bearing by its free end portion on a counter-element 53 in the grip 2.

In the figures the back portion 6 is shown rotatably connected to the grip 2 in a rotary connection 55 placed at a top portion of the grip 2. A person skilled in the art will appreciate that the point of rotation of the adjustable back portion 6 may be positioned elsewhere, for example at the bottom portion of the grip 2, and that the adjustable back portion 6 may be provided with several rotatable portions.

In alternative embodiments (not shown), the grip of the instrument may be provided with adjustment possibilities also at a belly portion of the grip 2 or at one or both side portions.

In the FIGS. 3 and 4 the actuator 3 has been moved to its inner or activated position, and the effector 9 in the form of needle-nose pliers, is in its closed position.

In the FIGS. 5-7 the adjustable back portion 6 is substantially moved into the grip 2 so that the grip 2 takes its near-least projecting position and the distance between the actuator 3 and the back portion 6 will be near to the smallest possible, as the claw member 47 has been moved into bearing across the outermost but one dog 48. The back portion 6 of the grip 2 may be secured in a number of intermediate positions between the position shown, for example, in FIG. 5 and the position shown, for example, in FIG. 4. Such a possibility of adjustment is important in order to achieve a best possible adaptation of the grip 2 to the surgeon's hand size, so that the comfort of use will be optimal.

In the FIGS. 5 and 7 the connection switch 26 for the ratchet mechanism is placed in the armed position and the pawl 24 is engaged with the ratchet bar 22. In FIG. 5 the actuator 3 is in its initial position, whereas in FIG. 7 the actuator 3 has been moved partly in towards it innermost position. Due to the ratchet mechanism having been activated by the slanting teeth of the pawl 24 meshing with the slanting teeth of the ratchet bar 22, the actuator 3 cannot be returned to its initial position as long as the ratchet mechanism is activated. However, the actuator 3 allows itself to be pushed further in towards its innermost position through the application of a driving force in the opposite direction exceeding the biasing force exerted by the spring 35. Due to the flexible element 28, the ratchet bar 22 may be moved in one direction relative to the pawl 24 by the slanting teeth projecting from the ratchet bar 22 being allowed to slide over the complementary teeth projecting from the pawl. Thus the effector 9 may be placed into practically any position between the fully open and fully closed position and secure tissues or clamp blood vessels without the risk of the effector 9 opening in an uncontrolled manner. Applying further force on the actuator 3 carries out further clamping.

The way the instrument is formed in the present exemplary embodiment, the linkage 5 and the articulated connection of the actuator 3 thereto and to the grip 2 will provide non-linear transmission between the actuator 3 and the effector 9. The non-linear transmission represents very important advantages with respect to both the surgeon's muscle use and the control of the effector 9 and involves that the effective force of the effector 9, which is transmitted from an external force applied to the actuator 3, is increased continuously from when this is in its non-activated position and until the actuator 3 is in its fully activated position, in which the effector 9 is preferably closed. This is due to the fact that the transmission between the movement of the actuator 3 and the movement of the effector 9 decreases as the actuator 3 is moved from a non-activated position into a fully activated position.

In the FIGS. 8-11 the ratchet mechanism and its connection to the connection switch 26 via the flexible element 28 are shown on a larger scale. In FIG. 8 is shown the situation described above in connection with the description of FIG. 5. In FIG. 9 is shown a perspective view of a portion of the instrument 1 as the actuator 3 has been moved to a substantially fully activated position. When the actuator 3 is moved from the position as shown, for example, in FIG. 8, to the position as shown in FIG. 9, while at the same time the connection switch 26 is in the activated position, the slanting teeth of the ratchet bar 22 will be moved relative to the slanting teeth of the pawl 24 while, at the same time, the pawl 24 is biased towards the ratchet bar 22 by means of the flexible element 28. The moment that the external force, which is applied to the actuator 3, ceases or at least is smaller than the biasing force from the spring 35 (see for example FIG. 7), the spring 35 will urge the teeth of the ratchet mechanism into full engagement and lock the effector 9 against moving into the open position.

In FIG. 10 the connection switch 26 has been moved into the non-activated position while the teeth of the pawl 24 still engage the teeth of the ratchet bar 22. In this situation, the flexible element 28 is in a somewhat stretched form, which entails that the pawl 24 is subjected to tensile force acting in the upward direction. However, the frictional forces occurring between the teeth of the bearing surfaces of the pawl 24 and ratchet bar 22 will prevent the pawl 24 from rotating clockwise about the axle 25. To reduce the frictional forces in such a way that the tensile force from the flexible element 28 pulls the pawl 24 out of the engagement with the ratchet bar 22, the bearing pressure of the teeth on each other is reduced by subjecting the actuator 3 to a small pressure force or inward movement. The teeth of the pawl 24 will then be pulled out of engagement with the teeth of the ratchet bar 22, as shown in FIG. 11. It should be noted that in FIG. 11 the ratchet bar 22 is moved further than what is necessary to disengage the pawl 24 from the ratchet bar 22.

The slant of the set of teeth of the ratchet mechanism 20 is decisive for whether the spring 35 is necessary in order to maintain the engagement of the sets of teeth of the ratchet bar 22 and pawl 24 after the connection switch 26 has been moved into the non-activated position. In embodiments (not shown), in which the teeth are not slanting, the engagement of the teeth will cease as soon as the connection switch 26 is moved into the non-activated position. In such a case it will also be possible, by means of the actuator 3, to drive a ratchet bar in both directions relative to a pawl engaging the ratchet bar.

A person skilled in the art will appreciate that the actuator 3, which is shown in the present exemplary embodiment with a so-called closed finger-grip, may be provided with a so-called open finger-grip.

In an alternative embodiment (not shown), the movable connection switch 26 is replaced with a press switch of a type known per se, preferably placed in a natural position at the upper portion of the grip relative to the positioning of the fingers about the grip.

1.-10. (canceled)

11. A laparoscopic instrument comprising a grip provided with an actuator which is arranged to effect, through a linkage, the manipulation of an effector placed in a first end portion of a tubular element, the tubular element extending at its second end portion into the grip of the instrument and being connected to a portion of the linkage, the linkage including a ratchet bar which is arranged to cooperate with a substantially complementary pawl, as slanting teeth of the pawl are arranged to be activated or deactivated for engagement with slanting teeth of the ratchet bar by means of a connection switch, wherein the connection switch is connected to the pawl via a flexible element which is arranged to bias the pawl into engagement with the ratchet bar when the connection switch is in a first position, and which is arranged to preload a pull on the pawl when the connection switch is in a second position, the pawl being arranged to be disengaged from the ratchet bar only when the pull from the flexible element at least exceeds an opposite resultant of the frictional forces occurring between the slanting teeth of the pawl and the ratchet bar, and which could maintain said engagement even after the connection switch has been moved from the first position into the second position.

12. The laparoscopic instrument in accordance with claim 11, wherein the flexible element connecting the pawl to the connection switch, is provided with a curved portion.

13. The laparoscopic instrument in accordance with claim 12, wherein the biasing force of the flexible element acts substantially perpendicularly to an interface between the ratchet bar and the pawl.

14. The laparoscopic instrument in accordance with claim 12, wherein the connection switch and the actuator are arranged to be operated independently of each other.

15. The laparoscopic instrument in accordance with claim 12, wherein a biasing device is arranged to exert a force which will move, after the connection switch has been deactivated and the actuator has been pressed inwards by such an amount that the flexible element has been able to pull the pawl out of engagement with the ratchet bar, the actuator into its initial position as long as an external force which is applied to the actuator is smaller than the force from the biasing device.

16. The laparoscopic instrument in accordance with claim 12, wherein the grip is provided with at least one adjustable portion, so that the grip can be size-adjusted to different user requirements.

17. The laparoscopic instrument in accordance with claim 11, wherein the biasing force of the flexible element acts substantially perpendicularly to an interface between the ratchet bar and the pawl.

18. The laparoscopic instrument in accordance with claim 17, wherein the connection switch and the actuator are arranged to be operated independently of each other.

19. The laparoscopic instrument in accordance with claim 17, wherein a biasing device is arranged to exert a force which will move, after the connection switch has been deactivated and the actuator has been pressed inwards by such an amount that the flexible element has been able to pull the pawl out of engagement with the ratchet bar, the actuator into its initial position as long as an external force which is applied to the actuator is smaller than the force from the biasing device.

20. The laparoscopic instrument in accordance with claim 17, wherein the grip is provided with at least one adjustable portion, so that the grip can be size-adjusted to different user requirements.

21. The laparoscopic instrument in accordance with claim 11, wherein the linkage is arranged to provide non-linear transmission between the actuator and the effector.

22. The laparoscopic instrument in accordance with claim 21, wherein the connection switch and the actuator are arranged to be operated independently of each other.

23. The laparoscopic instrument in accordance with claim 21, wherein a biasing device is arranged to exert a force which will move, after the connection switch has been deactivated and the actuator has been pressed inwards by such an amount that the flexible element has been able to pull the pawl out of engagement with the ratchet bar, the actuator into its initial position as long as an external force which is applied to the actuator is smaller than the force from the biasing device.

24. The laparoscopic instrument in accordance with claim 21, wherein the grip is provided with at least one adjustable portion, so that the grip can be size-adjusted to different user requirements.

25. The laparoscopic instrument in accordance with claim 11, wherein the connection switch and the actuator are arranged to be operated independently of each other.

26. The laparoscopic instrument in accordance with claim 25, wherein a biasing device is arranged to exert a force which will move, after the connection switch has been deactivated and the actuator has been pressed inwards by such an amount that the flexible element has been able to pull the pawl out of engagement with the ratchet bar, the actuator into its initial position as long as an external force which is applied to the actuator is smaller than the force from the biasing device.

27. The laparoscopic instrument in accordance with claim 25, wherein the grip is provided with at least one adjustable portion, so that the grip can be size-adjusted to different user requirements.

28. The laparoscopic instrument in accordance with claim 11, wherein the connection switch and the actuator are placed ergonomically relative to the positions of the fingers on the instrument, so that the instrument is arranged to be manipulated without the user having to move the fingers from another location on the instrument.

29. The laparoscopic instrument in accordance with claim 28, wherein a biasing device is arranged to exert a force which will move, after the connection switch has been deactivated and the actuator has been pressed inwards by such an amount that the flexible element has been able to pull the pawl out of engagement with the ratchet bar, the actuator into its initial position as long as an external force which is applied to the actuator is smaller than the force from the biasing device.

30. The laparoscopic instrument in accordance with claim 28, wherein the grip is provided with at least one adjustable portion, so that the grip can be size-adjusted to different user requirements.

31. The laparoscopic instrument in accordance with claim 11, wherein a biasing device is arranged to exert a force which will move, after the connection switch has been deactivated and the actuator has been pressed inwards by such an amount that the flexible element has been able to pull the pawl out of engagement with the ratchet bar, the actuator into its initial position as long as an external force which is applied to the actuator is smaller than the force from the biasing device.

32. The laparoscopic instrument in accordance with claim 31, wherein the grip is provided with at least one adjustable portion, so that the grip can be size-adjusted to different user requirements.

33. The laparoscopic instrument in accordance with claim 11, wherein the biasing device is constituted by a spring, which is placed between and attached to a portion of the linkage and a portion of the grip.

34. The laparoscopic instrument in accordance with claim 33, wherein the grip is provided with at least one adjustable portion, so that the grip can be size-adjusted to different user requirements.

35. The laparoscopic instrument in accordance with claim 11, wherein the grip is provided with at least one adjustable portion, so that the grip can be size-adjusted to different user requirements.

36. The laparoscopic instrument in accordance with claim 35, wherein one of the at least one adjustable portion is placed in a back portion of the grip.
